(19) United States
(12) Patent Application Publication
(10) Pub. No.: US 2009/0222081 A1
(43) Pub. Date: Sep. 3, 2009

(54) METHODS AND APPARATUS FOR REDUCING VALVE PROLAPSE

(75) Inventors: Richard J. Linder, Sandy, UT (US); Scott D. Miles, Sandy, UT (US); Daryl R. Edmiston, Draper, UT (US); Clark C. Davis, Holladay, UT (US)

Correspondence Address: Holland & Hart LLP/Coherex Medical, 60 East South Temple, Suite 2000, Salt Lake City, UT, 84111, US

(73) Assignee: COHEREX MEDICAL, INC., Salt Lake City, UT (US)

(21) Appl. No.: 12/359,185

(22) Filed: Jan. 23, 2009

Related U.S. Application Data
(60) Provisional application No. 61/023,368, filed on Jan. 24, 2008.

(51) Int. Cl. A61F 2/24

(52) U.S. Cl. 623/2.1

(57) ABSTRACT

Medical devices, systems and methods for treating valve prolapse in, for example, the mitral valve. The medical device is employed by delivering the device percutaneously and lodging the device adjacently above the valve. With this arrangement, the device provides a back-stop to prevent valve prolapse and, thus, prevent valve regurgitation.

### CROSS-REFERENCE TO RELATED APPLICATIONS

The present application claims the benefit of U.S. Provisional Patent Application Ser. No. 61/023,368, filed Jan. 24, 2008, entitled METHODS AND APPARATUS FOR REDUCING VALVE PROLAPSE, the disclosure of which is incorporated by reference herein in its entirety.

### TECHNICAL FIELD

The present invention relates generally to methods and devices for limiting valve regurgitation. More specifically, the present invention relates to medical devices and methods for implanting medical devices percutaneously to reduce or limit valve prolapse and regurgitation.

### BACKGROUND

The human heart generally includes four valves. Of these valves, a most critical one is known as the mitral valve. The mitral valve is located in an opening between the left atrium and left ventricle. The mitral valve acts as a check valve and is intended to prevent regurgitation of blood from the left ventricle into the left atrium when the left ventricle contracts. In preventing blood regurgitation the mitral valve must be able to withstand considerable back pressure as the left ventricle contracts.

The valve cusps or leaflets of the mitral valve are anchored to the muscular wall of the heart by delicate but strong fibrous cords so as to support the cusps during left ventricular contraction. In a healthy mitral valve, the geometry of the mitral valve ensures that the cusps overlie each other to preclude regurgitation of the blood during left ventricular contraction.

Many known methods for treating mitral regurgitation resort to open heart surgery, typically by implanting artificial valves. Such procedures are expensive, are extremely invasive requiring considerable recovery time and, most significantly, pose mortality risks. Further, such open heart procedures are particularly stressful on patients whom already have a cardiac condition. As such, open heart surgery is typically reserved as a last resort and is usually employed late in the mitral regurgitation progression. Moreover, the effectiveness of such procedures is difficult to assess during the procedure and may not be known until a much later time. Therefore, the ability to make adjustments or modifications to the prostheses in order to obtain optimum effectiveness is extremely limited. Later corrections, if made at all, require still another open heart surgery bringing all of the risks and disadvantages discussed previously.

Based on the foregoing, it would be advantageous to employ a less invasive procedure to treat mitral regurgitation or any other valve regurgitation issues.

### BRIEF SUMMARY OF THE INVENTION

Embodiments of the present invention are directed to medical devices, systems and methods for implanting a medical device into the heart to minimize valve prolapse. In one embodiment, the medical device may include a loop portion and an intermediate portion. The loop portion includes an outer periphery and an inner periphery, the outer periphery being configured to be positioned above the valve of the heart and lodged against tissue of the heart. The intermediate portion is configured to extend from the inner periphery of the loop portion and configured to substantially minimize or limit upward movement of leaflets of the valve.

In another embodiment, the loop portion is configured to self expand and bias against heart tissue adjacently above a valve annulus. The device may also include tines extending from an outer periphery of the loop portion. Further, the device may include a tissue growth member promote tissue growth therein. In another embodiment, the loop portion and the intermediate portion can exhibit a substantially flat shape.

In another embodiment, the loop portion includes a tubular configuration, the outer periphery of the tubular configuration being sized and configured to lodge against tissue at a lower portion of the left atrium with the intermediate portion extending from a lower portion of the tubular configuration.

In still another embodiment, the intermediate portion includes multiple intermediate portions extending between different portions of the loop portion. The intermediate portion may also include multiple lines extending in a first direction from the inner periphery and multiple lines extending in a second direction from the inner periphery. With this arrangement, the first direction is substantially transverse to the second direction.

In another embodiment, the intermediate portion exhibits a coiled configuration so as to allow blood flow therethrough while still substantially preventing upward movement of the leaflets. Further, in another embodiment, the intermediate portion exhibits a curved or arcuate configuration to provide a back-stop to the leaflets of the valve.

In another embodiment of the present invention, the medical device includes a tubular portion and an intermediate portion. The tubular portion includes an outer periphery and a lower portion, the outer periphery being configured to be lodged against tissue of the heart adjacently above an annulus of the valve. The intermediate portion is configured to extend from the lower portion of the tubular portion and is configured to extend over leaflets of the valve to substantially minimize or limit upward movement of the leaflets of the valve.

In another embodiment, the tubular portion includes a tissue growth member configured to promote tissue in-growth therein and help permanently attach the tubular portion in the heart. Further, in still another embodiment, the medical device may include tines at the outer periphery of the tubular portion, the tines being configured to lodge the tubular portion against the tissue of the heart. In another embodiment, the tubular portion includes an upper loop portion and a lower loop portion with intermediate extensions therebetween to define the tubular portion.

In another embodiment, the intermediate portion includes multiple intermediate portions extending between different portions of the lower portion of the tubular portion. Further, the intermediate portion can include a first set of multiple lines extending in a first direction from the lower portion of the tubular portion and a second set of multiple lines extending in a second direction from the lower portion of the tubular portion such that the first direction is substantially transverse to the second direction.

In another embodiment, the tubular portion is configured to self expand and bias against the tissue of the heart adjacently above the valve.

In still another embodiment of the present invention, the medical device includes an intermediate portion, an anchor portion and a plurality of tabs. The intermediate portion is configured to be positioned above a leaflet free-edge of the valve. The anchor portion is configured to extend from the intermediate portion and configured to lodge against heart tissue above the valve. The plurality of tabs are coupled to the intermediate portion and are configured to abut against leaflets of the valve and substantially minimize or limit upward movement of the leaflets of the valve. In another embodiment, the multiple tabs extend downward and outward from opposite sides of the intermediate portion.

In accordance with another embodiment of the present invention, a method is provided for reducing valve prolapse. The method includes disposing a frame within the heart adjacent a valve and limiting movement of at least one leaflet of the valve with a portion of the frame.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is a profile view of a medical device system, according to one embodiment of the present invention;

FIG. 1A is a partial cross-sectional view of a portion of the medical device system of FIG. 1, depicting a medical device in a constrained position within a distal portion of a catheter, according to another embodiment of the present invention;

FIGS. 2A and 2B are cross-sectional views of a heart, depicting the medical device of FIG. 1 being deployed above the mitral valve in the heart;

FIGS. 3A through 3C are respective top views of various embodiments of the medical device depicted in FIG. 2B;

FIGS. 4A and 4B are respective top views of additional embodiments of the medical device depicted in FIG. 2B, according to the present invention;

FIGS. 5 and 5A are perspective and top views, respectively, of a medical device, according to another embodiment of the present invention;

FIG. 5B is a top view of another embodiment of the medical device of FIG. 5, according to the present invention;

FIGS. 6 and 6A are perspective and top views, respectively, of a medical device, according to another embodiment of the present invention;

FIG. 6B is a top view of another embodiment of the medical device of FIG. 6, according to the present invention;

FIG. 7 is a cross-sectional view of a heart with a medical device implanted therein according to an embodiment of the present invention;

FIG. 7A is a top view of the medical device of FIG. 7;

FIG. 8 is a profile view of the left side of the heart, depicting a medical device being deployed below the mitral valve to effectively shorten cordae in the heart, according to another embodiment of the present invention;

FIGS. 8A and 8B are enlarged side views of the medical device of FIG. 8 being deployed and attached to the cordae, according to another embodiment of the present invention;

FIG. 9 is an enlarged side view of a medical device, depicting the medical device being used to effectively shorten cordae in the heart, according to another embodiment of the present invention;

FIG. 10 is a perspective view of a medical device for implanting within the left ventricle, according to another embodiment of the present invention; and

FIGS. 10A and 10B are side views of the medical device of FIG. 10 being deployed within the left ventricle of the heart, according to anther embodiment of the present invention.

### DETAILED DESCRIPTION OF THE INVENTION

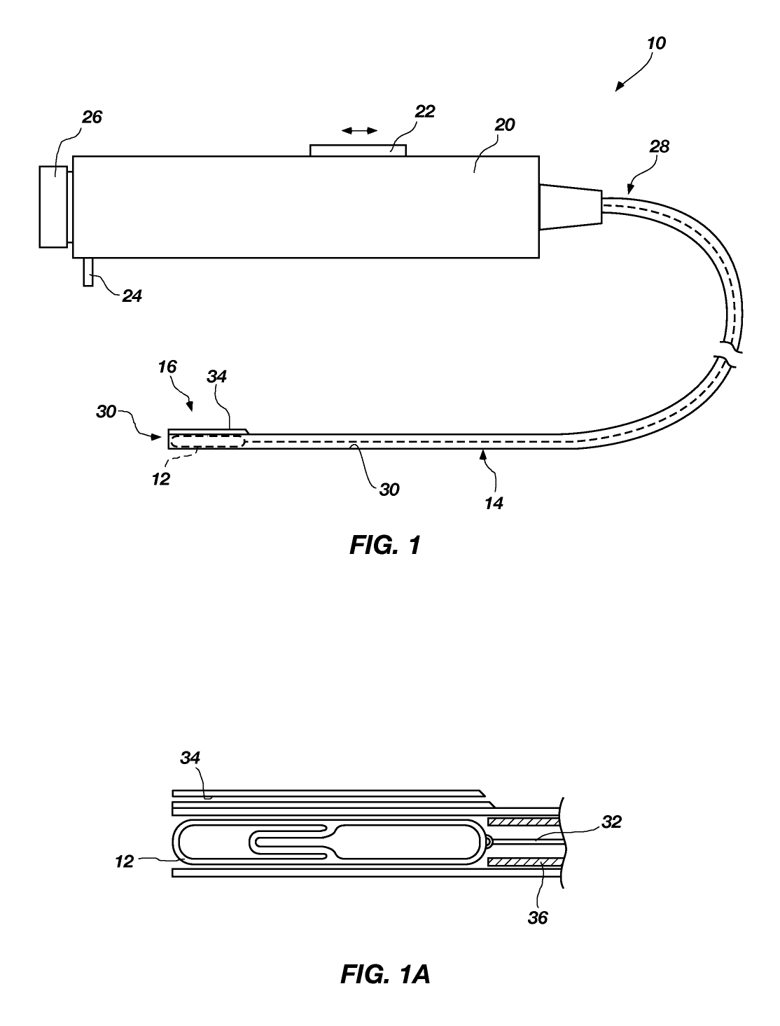

Referring to FIGS. 1 and 1A, a medical device system 10 is shown that may be used for advancing a medical device 12 that is configured to be employed to substantially prevent valve prolapse in, for example, the mitral valve. In particular, a distal portion 16 of a catheter 14 of the medical device system 10 may be advanced into the left atrium to implant the medical device 12 adjacent to and around the atrioventricular valve annulus in the left atrium.

The medical device system 10 may include a handle 20 having an actuator 22, a fluid port 24 and a disengagement portion 26. Further, the medical device system 10 may include a catheter 14 having a proximal portion 28 and a distal portion 16 with a catheter lumen 30 extending therethrough. The proximal portion 28 of the catheter 14 is coupled at a distal end of the handle 20. The distal portion 16 of the catheter 14 may be sized and configured to hold and maintain the medical device 12 when advancing the medical device to the mitral valve in the heart. Further, the medical device 12 may be coupled to the medical device system 10, within the lumen 30, via one or more lines or tethers 32. The tether(s) 32 can extend through the catheter 14 and can be coupled to the handle 20. The tether(s) 32 may further be releasable via the disengagement portion at the handle 20. Such a medical device system 10 can be advanced to the left atrium via a guide wire (not shown) by advancing the distal portion 16 of the catheter 14 over the guide wire through a rapid exchange (Rx) lumen 34 subsequent to the guide wire being properly advanced to the heart. Disclosure of a tethering system as well as the rapid exchange lumen can be found in Applicant's pending patent application, application Ser. No. 11/836,051, the disclosure of which is incorporated herein in its entirety. It should be noted that the medical device system 10 can also be configured to facilitate over the wire delivery, as known in the art.

With respect to FIG. 1A, an enlarged view of the medical device 12 is shown at the distal portion 16 of the catheter 14 (shown in cross-section) in a constrained configuration. The catheter 14 may also include a coil 36 positioned proximally the medical device 12 with the tether 32 extending through or along side the coil 36. The coil 36 may extend through the catheter lumen 30 to the handle 20 and is configured to either push the medical device 12 from the catheter 14 while the catheter remains fixed or hold the medical device 12 in a substantially fixed position while the catheter 14 is moved proximally via the actuator 22 to, thereby, deploy the medical device 12 from the catheter 14.

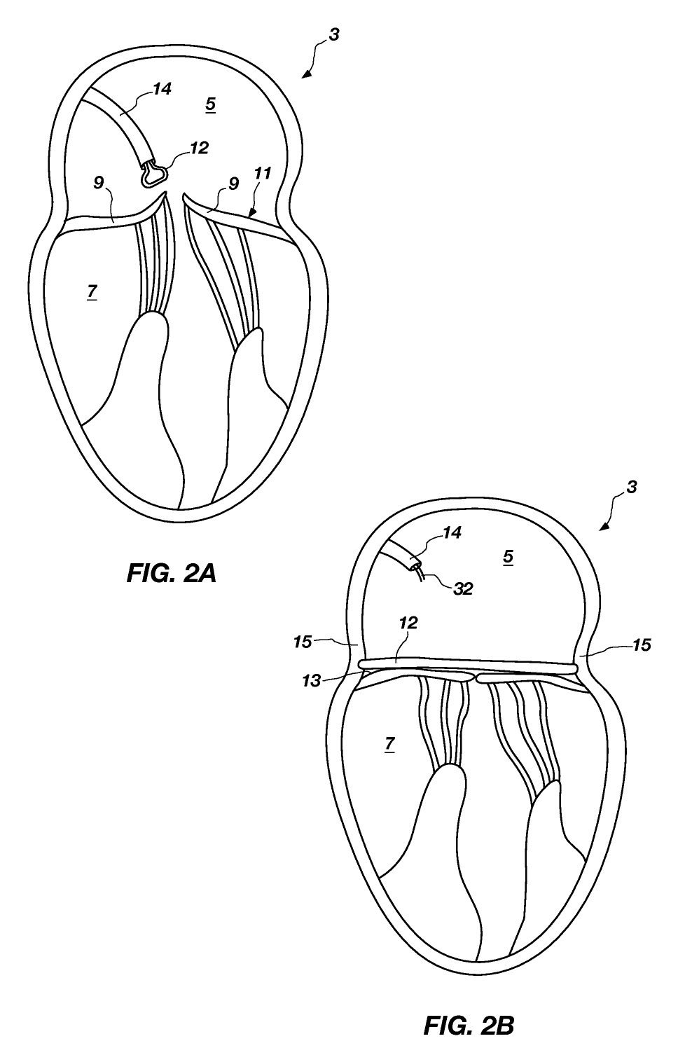

Referring to FIGS. 2A and 2B, there is depicted a side profile view of a left portion of a heart 3, including a defect in the mitral valve 11 causing regurgitation between the left atrium 5 and the left ventricle 7. In particular, FIG. 2A depicts leaflets 9 of the mitral valve 11 in a prolapsed state, allowing blood back-flow back into the left atrium 5 (the defect).

The catheter 14 may be advanced into the left atrium 5, for example, via a transseptal puncture through the septum wall. The physician can utilize imaging techniques to determine a desired position to begin deployment of the medical device 12. Once the catheter 14 is in the desired position, the medical device 12 may be deployed from the catheter 14 and may be positioned at a lower portion of the left atrium 5, for example above and adjacent to the mitral valve 11 or around the atrioventricular valve annulus 13 in the left atrium 5, as depicted in FIG. 2B. When deployed from the catheter 14, the medical device 12 may be configured to self expand and bias against the tissue wall 15 of the left atrium 5. Once confirmation that the medical device 12 is in a desired position and properly engaged against the tissue wall 15, the one or more tethers 32 may be disengaged from the medical device 12 and the catheter 14 may be withdrawn from the left atrium 5. With this arrangement, the periphery of the medical device 12 is configured to be positioned against the valve annulus 13 such that the medical device 12 provides a back-stop (not shown) over the leaflets 9 to, thereby, substantially prevent valve prolapse and the associated mitral regurgitation. The medical device 12 and “back-stop” are described in further detail hereafter.

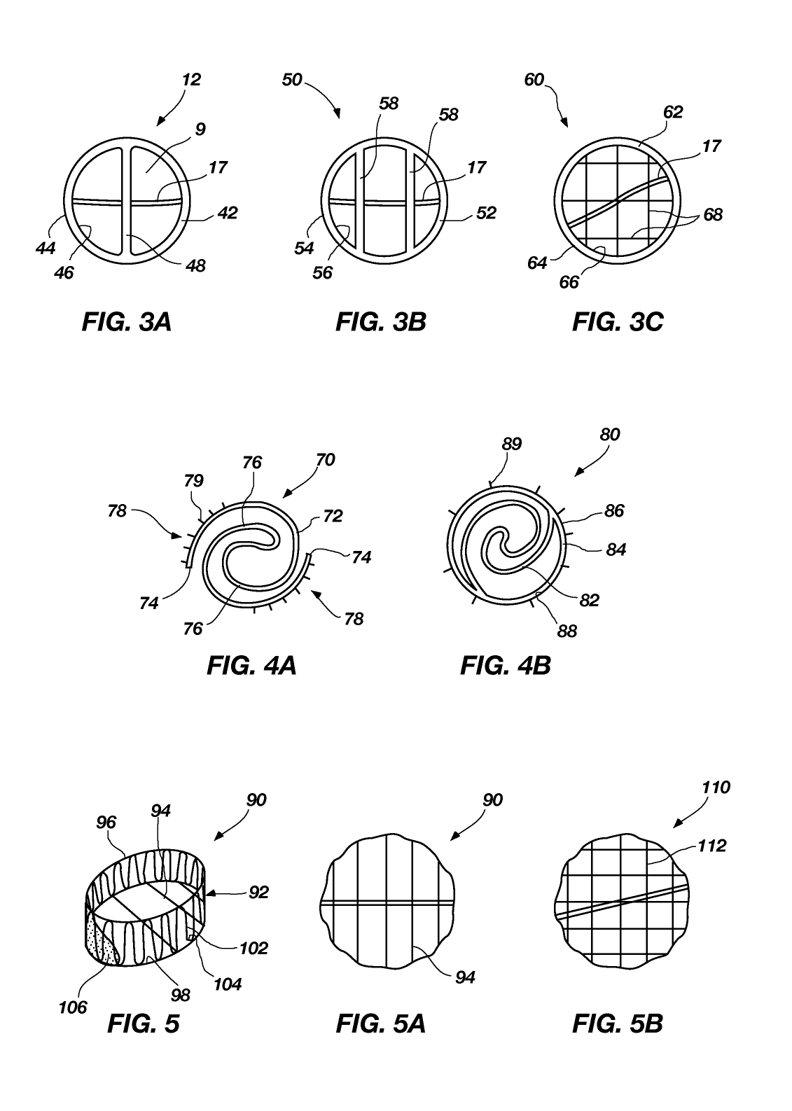

Referring now to FIGS. 3A through 3C, various embodiments of the medical device, such as depicted in FIG. 2B, are illustrated with the medical devices being shown from a top view with the leaflets in a coapted position. Each of these embodiments of the medical device may be substantially flat or planar when in their relaxed fully expanded position. However, when lodged adjacently above the mitral valve, the medical device may flex and be slightly out of plane, but still exhibit a substantially flat shape or geometry.

With respect to FIG. 3A, in one embodiment, the medical device 12 may include a looped frame 42 for implanting over the mitral valve. The looped frame 42 may be a circular shaped, oval shaped or any other suitable geometry for disposal over the mitral valve. The looped frame 42 may include an outer periphery 44 and an inner periphery 46. The inner periphery 46 of the looped frame 42 may define an interior space with a single intermediate cross-member 48 extending across a central portion of the interior space of the looped frame 42. With this structure, it is most advantageous to orient the looped frame so that the intermediate cross-member 48 is transverse to a free edge 17 of the leaflets 9. As previously set forth, the medical device 12 includes a low profile having a substantially flat shape (see FIG. 2B) such that the cross-member 48 is configured to extend longitudinally over the free edge 17 of the leaflets 9 of the valve perpendicularly or at a desired angle relative thereto. In this manner, when the valve is in an open position (not shown), blood can pass into the left ventricle. When the valve is intended to be in a closed position, the cross-member 48 overcomes the defect and substantially prevents the valve leaflets 9 from extending upward with the cross-member 48 sitting adjacently against the leaflets 9 and acting as a back-stop for the valve leaflets 9 in the closed position.

With respect to FIG. 3B, another embodiment of a medical device 50 is provided. Such medical device 50 can also include a looped frame 52 including an outer periphery 54 and an inner periphery 56, similar to the previous embodiment, except in this embodiment, the medical device 50 can include multiple cross-members 58 or struts extending across an interior space defined by the inner periphery 56 of the looped frame 52. Similar to the previous embodiment, it may be desired to orient this device so that the cross-members 58 are transverse to, or at an acute angle relative to, the leaflet free edge 17, such as depicted in FIG. 3B.

FIG. 3C illustrates another embodiment of a medical device 60, similar to the previous embodiments, including a looped frame 62 having an outer periphery 64 and an inner periphery 66. In this embodiment, the looped frame 62 may include multiple cross-members or lines 68 extending transverse to each other. In particular, within an interior space defined by the inner periphery 66 of the looped frame 62, there can be multiple, substantially parallel lines 68 or cross members extending in a first direction and multiple parallel lines 68 or cross members extending in a second direction, the first direction being substantially transverse to the second direction to provide a screen like configuration. In another embodiment, the lines 68 may extend within the interior space, connected to the looped frame 62 at opposing sides, but extend in a non-parallel configuration. In either case, this embodiment allows the medical device 60 to be positioned without a specific orientation with respect to the leaflet free edge 17. In each of these embodiments, the cross-members or lines provide a permanent back-stop to the valve leaflet of, for example, the mitral valve.

Referring now to FIGS. 4A and 4B, other embodiments of a medical device that can be implanted, similar to the shown in FIGS. 2A and 2B, are disclosed. With respect to FIG. 4A, the medical device 70 may include a frame 72 with a circular structure or curved structure having two free ends 74 with an intermediate portion 76 therebetween. The intermediate portion 76 extending between the two free ends 74 may include a coiled or semi-coiled configuration. The two free ends 74 include free end portions 78 that can bias outward against the tissue to assist in lodging the medical device 70 in proper position. Further, the free end portions 78 may include tines 79 to facilitate anchoring the medical device 70 within the heart valve. The intermediate portion 76 least is sized and configured to have at least a portion thereof positioned adjacently above the leaflets of a valve so as to provide a back-stop to substantially prevent regurgitation. Further, as in the previous embodiments, the frame 72 may be sized and configured to be readily collapsed or pulled into and disposed out of a catheter (not shown) for delivery and deployment adjacent the mitral leaflets in the left atrium. It is also contemplated that the medical device 70 of this embodiment can be employed with a single free end, rather than the two free ends.

FIG. 4B discloses another embodiment of a medical device 80 with an intermediate portion 82 similar to the previous embodiment, except in this embodiment there are no free ends. In particular, the medical device 80 of this embodiment includes a looped frame 84 having an outer periphery 86 and an inner periphery 88 with an intermediate portion 82 extending within an interior space defined substantially by the inner periphery 88 of the looped frame 84. As in previous embodiments, the frame 84 may include tines 89 to anchor within the tissue adjacently above a valve, such as a mitral valve. The intermediate portion 82 may exhibit a coiled configuration sized and configured to be positioned above leaflets of a valve to resist vertical movement of the leaflets and, thereby, provide a back-stop for the leaflets of the valve.

Referring now to FIGS. 5 and 5A, another embodiment of a medical device 90 is provided. The medical device 90 of this embodiment may include a tubular structure 92 with multiple lines 94 extending across a lower portion of the tubular structure 92. The tubular structure 92 may include an upper looped frame 96 and a lower looped frame 98 with an intermediate extension 102 configured to extend between the upper looped frame 96 and lower looped frame 98 in, for example, a sinusoidal or undulating configuration, or any other suitable configuration to form a tubular structure between the upper looped frame 96 and lower looped frame 98. The lower looped frame 98 may include, or be coupled to, the multiple lines 94 or struts extending across the lower looped frame 98 such that the lines 94 extend between different points of the lower looped frame 98. In one embodiment, the lines 94 may extend parallel with respect to each other. In another embodiment the lines 94 may extend in a non-parallel configuration.

In another embodiment, the medical device 90 can be formed with one or neither of the upper and lower looped frames. As such, the lines 94 may extend between the lower portion of loops 104 of the sinusoidal or undulating configuration. In this manner, the lines 94 extending across the lower portion of the medical device 90 are sized and configured to act as a back-stop for leaflets of a valve. The medical device 90 of this embodiment may be configured to self expand when delivered, similar to the previously described embodiments, or they may be configured to be expanded over an inflatable balloon or other expansion device, such as known in the art of deploying tubular stents, to implant such device adjacently above a valve.

The medical device 90 may also include a tissue growth member 106 disposed over or weaved between the upper looped frame 96 and lower looped frame 98 of the medical device 90. Such a tissue growth member 106 may permanently attach the medical device 90 to the tissue in the heart while leaving the lines 104 exposed to provide the previously described backstop to prevent valve prolapse. The tissue growth member 106 may be a porous member made from a polymeric or metallic material, such as fabric, felt, Dacron, polyurethane, Nitinol weaves or braids, or any other suitable polymeric or metallic materials configured to induce tissue in-growth, as known in the art.

With respect to FIG. 5B, another embodiment of a medical device 110 is disclosed. This embodiment can include structure similar to that described with respect to the previous embodiment, except additional lines 112 extending between the lower portion of the medical device 110 can extend transverse (or at some other desired angle) with respect to each other, similar to that described with respect to FIG. 3C. In this manner, the medical device 110 can be implanted adjacently above a valve such that the lines 112 provide a back-stop to substantially prevent valve prolapse in the valve leaflets and regurgitation of blood flow.

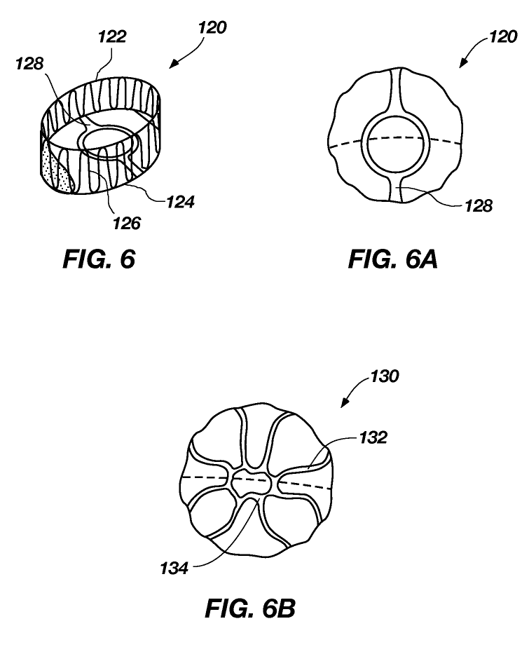

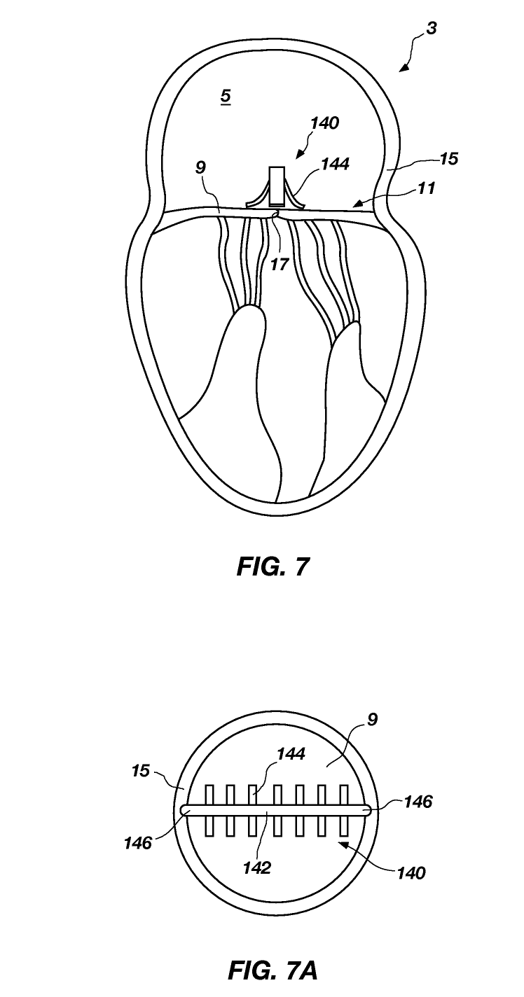

Other structural configurations can also be employed for a back-stop for a medical device. For example, FIGS. 6A and 6B, depict a tubular structure, generally similar to the medical device of FIG. 5A. In particular, the medical device 120 of this embodiment may include an upper looped frame 122 and a lower looped frame 124 with an intermediate portion 126 extending between the upper looped frame 122 and lower looped frame 124 to define the tubular structure. The lower looped frame 124 can include a lower extension portion 128 extending across an interior space defined by the lower looped frame. The lower extension portion 128 may include one or more minor extensions configured to interconnect to the lower looped frame 124 or lower portion of the intermediate portion 126 and interconnect to a back-stop. The back-stop can include a looped or circular portion and is positioned centrally such that the leaflets of a valve can contact at least two portions of the back-stop to, thereby, substantially prevent valve prolapse.

In another embodiment, as depicted in FIG. 6C, there is disclosed a medical device 130 having a tubular structure, similar to the previous embodiment, but with a differently configured back-stop. In particular, the back-stop can include multiple, generally u-shaped extensions 132 configured to extend from a lower portion of the medical device 130 toward a central portion of an interior space defined by the lower portion of the medical device. Each of the u-shaped extensions 132 may be interconnected to define a central portion 134 of the back-stop.

FIGS. 7 and 7A illustrate another embodiment of a medical device 140, deployed within the left atrium 5 of the heart 3 above the leaflets 9 of the mitral valve 11 (FIG. 7 being a rotated top view of the device). In this embodiment, the medical device 140 may include an intermediate portion 142 with multiple flexible tabs 144. The intermediate portion 142 may be an elongated member with opposing anchor ends 146 extending from the intermediate portion 142. The medical device 140 may be self expanding and provide a force, through the self expansion, to anchor itself within heart tissue. The opposing anchor ends 146 are configured to abut against the tissue wall 15 at a lower portion of the left atrium 5 and act as anchors to lodge the medical device 140 within the heart. The intermediate portion 142 is configured to be positioned and suspended adjacently above the mitral valve 11 and configured to be oriented and coincide with the opposed leaflet 9 free-edge 17 of the mitral valve 11. The multiple flexible tabs 144 extend laterally from the intermediate portion 142. Further, the multiple flexible tabs 144 extend from opposite sides of the intermediate portion 142 downwardly and laterally outward. Thus, the flexible tabs 144 may extend to a level below the intermediate portion 142 to contact the valve leaflets 9 and substantially prevent the valve leaflets to prolapse. In one embodiment, the tabs 144 may each be of a substantially common length and width. In another embodiment, the tabs 144 may exhibit different lengths, widths or both to provide different structural and flexible characteristics while biasing the leaflets of the valve. The tabs 144 are flexible and resilient and can be formed from a polymeric material or a metallic material, such as Nitinol. With this arrangement, the medical device can be lodged in a lower portion of the left atrium with multiple tabs 144 extending from an intermediate portion 142 of the medical device 140 to bias the leaflets 9 and act as a back-stop to the leaflets 9 of the mitral valve 11.

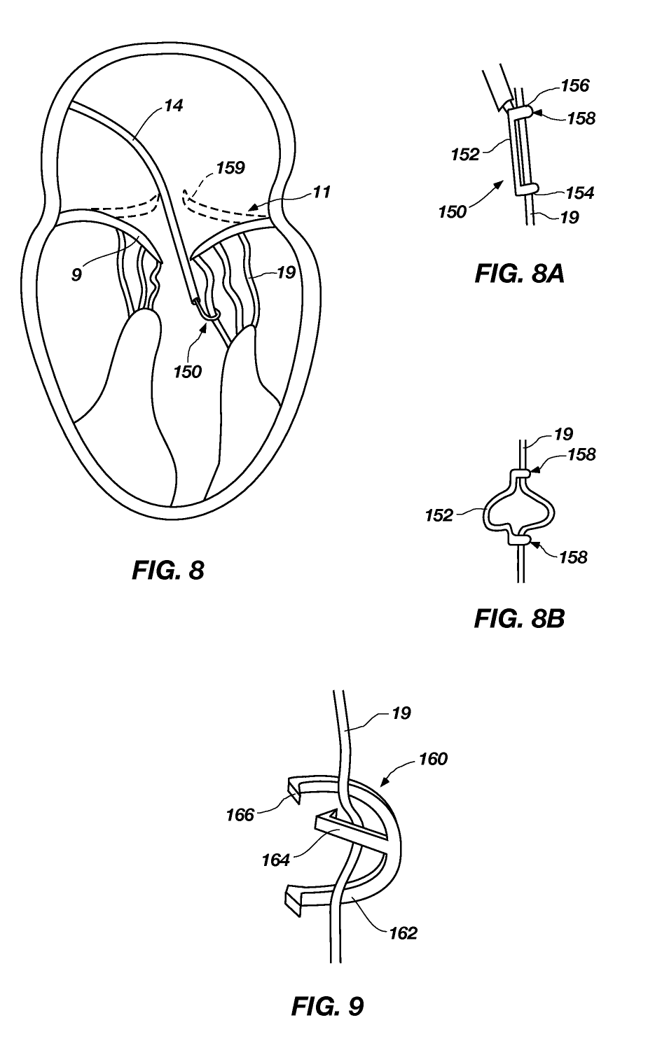

Referring now to FIG. 8, a medical device 150 is shown deployed via a catheter 14 over individual cordae 19 below the mitral valve 11, the mitral valve having the defect or prolapsed position 159 (leaflets shown in outline form in the defective position). As depicted in FIGS. 8A and 8B, the medical device 150 may include an intermediate portion 152 with a first end 154 and an opposite second end 156. The intermediate portion 152, when in a constrained position, is elongated and is configured to be disposed along side an individual cordae 19. The first end 154 and the second end 156 each include a grasping portion 158 configured to grasp a section of the cordae 19. Once the medical device 150 is placed over the cordae 19 with the grasping portions 158 fully engaged and grasping the cordae 19, and with the elongated intermediate portion 152 substantially fully extended in an elongate constrained position, the catheter 14 may disengage the medical device 150 from the constrained position and allow the intermediate portion 152 to move to an unconstrained position, as depicted in FIG. 8B. In the unconstrained position, the intermediate portion 152 moves to a non-elongate position and can curl or move outward. In other words, the first end 154 and second end 156 move closer to one another when the intermediate portion 152 transitions to an unconstrained or free state. With this arrangement, the cordae 19 is also moved together to pull slack from the cordae 19 so as to shorten an effective length of the cordae 19. Such a medical device 150 can be positioned on other cordae 19 until it is determined that the leaflets 9 are no longer in the prolapsed position 159.

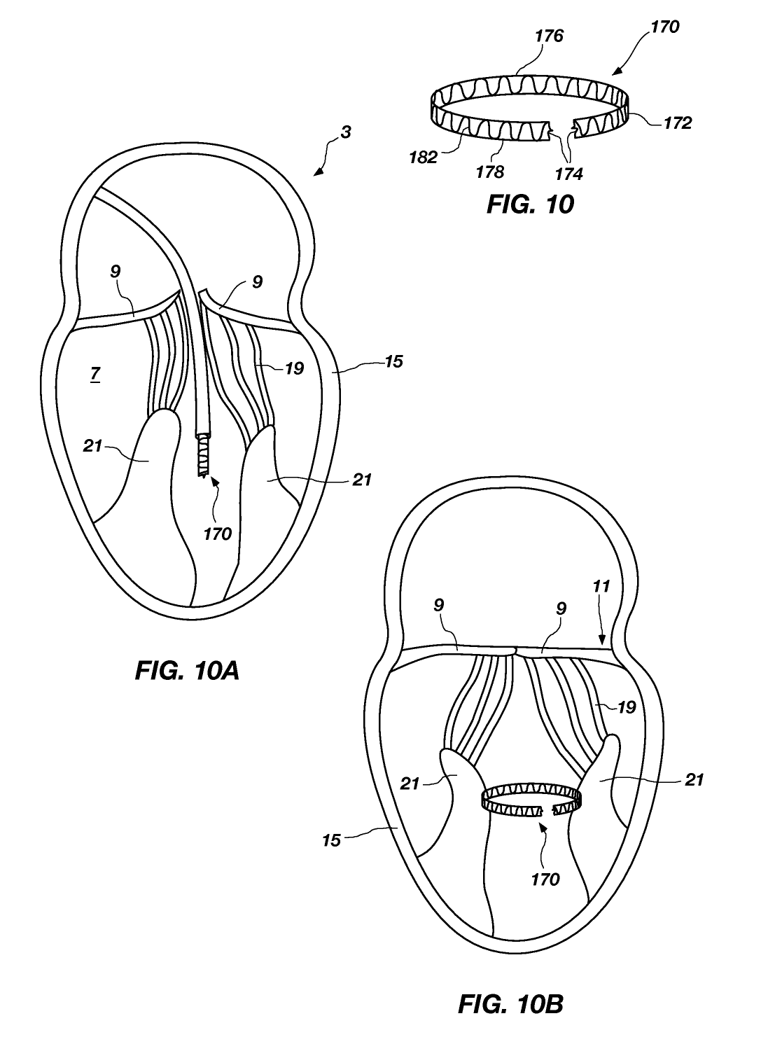

FIG. 9 discloses another embodiment of a medical device 160 to effectively shorten a length of the cordae 19 to substantially prevent valve prolapse. In particular, the medical device 160 may include a u-shaped portion 162 and a middle portion 164 forming an E-shaped configuration. When in the constrained position, the middle portion 164 is open (not shown) with respect to the u-shaped portion 162. In such open position, the medical device 160 is open to receive a cordae 19. The middle portion may them be displaced to an unconstrained position or a closed position, trapping the cordae 19 between the middle portion 164 and the u-shaped portion 162, as depicted. Each leg of the E-shaped configuration may include a nub 166 so as to substantially prevent the medical device 160 from becoming loose or migrating from the cordae 19. It is noted that while in the unconstrained position, the medical device 160 pulls the cordae 19 taut by removing slack in the cordae 19 to, thereby, effectively shorten the length or of the cordae 19 and substantially prevent valve prolapse.

FIG. 10 discloses another embodiment of a medical device 170 configured to shorten or otherwise remove the slack from the cordae and, thereby, enable the leaflets of the mitral valve to properly coapt. In the presently considered embodiment, the medical device 170 may include a frame 172 with an elongated configuration that can be heat-set into a circular or curved shape or loop with two opposite free ends 174. The medical device is sized and configured to be pulled into an elongated configuration when positioned within a catheter. In one embodiment, the medical device 170 may include a stent-like structure. Further, the medical device of the presently described embodiment may be self expanding and can move to a looped configuration (or some other suitable configuration) when unconstrained. The medical device 170 may include an upper portion 176 and a lower portion 178 with an intermediate extension 182 extending between the upper portion 176 and the lower portion 178. The upper portion 176 and the lower portion 178 may be formed from wire, such as Nitinol wire, each having free ends with the intermediate extension 182 being another wire weaved between and around the upper portion 176 and the lower portion 178. In another embodiment, the medical device 170 may be laser cut from a flat sheet of metal, such as Nitinol, and then heat set into the looped configuration.

Referring to FIGS. 10A and 10B, the medical device 170 depicted in FIG. 10 may be constrained within a catheter 14 and delivered into the heart 3 and specifically, into the left ventricle 7. The medical device 170 can then be deployed so as to abut partially against the papillary muscle 21 and the tissue wall 15 in the left ventricle 7 so as to displace the papillary muscle 21 and thereby remove the slack from the cordae 19 extending between the papillary muscle 21 and the leaflets 9 of the mitral valve 11. The medical device 170 may include tines (not shown) to assist the device in being lodged into, for example, the papillary muscle. Such a medical device 170 may also be positioned to abut against the cordae 19 and/or the papillary muscle 21 and/or the tissue wall 15. When deploying the medical device 170, imaging techniques may be employed, as known in the art, to determine if the medical device is properly positioned so that the leaflets 9 are not prolapsed or in a coapted position. If not positioned properly, the medical device 170 can be recaptured and then deployed again until the physician is satisfied with the position of the medical device 170. The medical device 170 may then completely disengaged from associated tethers and the catheter 14 can be withdrawn such as with previously described embodiments.

As known to one of ordinary skill in the art, the materials that may be employed for the various embodiments disclosed herein, as well as be compatible within the human anatomy, may include metals and/or polymers, such as, but not limited to, Nitinol, stainless steel, titanium, tantalum, chrome-moly steel, Teflon, silicon, polyester, polyethylene, polyurethane, acetal, nylon, polyamide, or any combinations thereof, or any other bio-compatible and/or bio-resorbable according to one or more of the variously described embodiments may be laser cut from flat sheets of Nitinol and manipulated into preferred configurations by heat-setting the medical device. The medical devices may also go through various polishing procedures, as known in the art. Further, radio opaque markers may be formed with or secured to the medical device, as known in the art for assistance in positioning the device within the heart. Additionally, it is contemplated that some materials or portions of the various embodiments disclosed herein can be formed from bioresorbable polymers, including polylactide, polyglycolide, poly-L-lactide, poly-DL-lactide, and various combinations thereof, and may be employed within, but not limited to, some of the anchors or tines disclosed herein.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes all modifications, equivalents, and alternatives falling within the spirit and scope of the invention as defined by the following appended claims. For example, the tissue growth member disclosed with respect to the medical device of FIGS. 5 and 6 can be employed in the medical devices disclosed in FIGS. 3A through 3C or any other of the medical devices disclosed herein. Similarly, the tines disclosed in FIGS. 4A and 4B can be employed on any of the other medical device embodiments to facilitate lodging the medical device in the heart tissue. Furthermore, while the detailed description has been disclosed as treating the problems of mitral regurgitation, the invention, as disclosed in the embodiments herein or any combinations/modifications thereof, can be employed to treat other valves within the human anatomy, such as the tricuspid valve, the aortic valve, the pulmonic valve, and any other valves within the human anatomy.

What is claimed is:

1. A medical device configured to be delivered with a catheter in a heart to minimize prolapse of a valve, comprising:
a loop portion having an outer periphery and an inner periphery, the loop portion configured to be positioned over the valve of the heart and lodged against tissue of the heart; and
an intermediate portion extending from the inner periphery of the loop portion and configured to substantially limit upward movement of leaflets of the valve.

2. The medical device of claim 1, wherein the loop portion is configured to self expand and bias against heart tissue adjacently above a valve annulus.

3. The medical device of claim 1, further comprising tines extending from an outer periphery of the loop portion.

4. The medical device of claim 1, wherein the intermediate portion comprises multiple intermediate portions extending between different portions of the loop portion.

5. The medical device of claim 1, wherein the intermediate portion comprises a first set of multiple lines extending in a first direction from the inner periphery and a second set of multiple lines extending in a second direction from the inner periphery, the first direction being transverse to the second direction.

6. The medical device of claim 1, wherein the intermediate portion exhibits a coiled configuration.

7. The medical device of claim 1, wherein the intermediate portion comprises an arcuate portion.

8. The medical device of claim 1, further comprising a member to promote tissue growth.

9. The medical device of claim 1, wherein the loop portion and the intermediate portion exhibit a substantially flat shape.

10. The medical device of claim 1, wherein the loop portion exhibits a tubular configuration, the outer periphery of the tubular configuration being sized and configured to lodge against tissue at a lower portion of the heart, the intermediate portion extending from a lower portion of the tubular configuration.

11. A medical device configured to be delivered with a catheter in a heart to minimize prolapse of a valve, comprising:
a tubular portion having an outer periphery and an lower portion, the outer periphery being sized and configured to be lodged against tissue of the heart adjacently above an annulus of the valve; and
an intermediate portion extending from the lower portion of the tubular portion and configured to extend over leaflets of the valve to limit upward movement of the leaflets of the valve.

12. The medical device of claim 11, wherein the tubular portion comprises a tissue growth member configured to promote tissue in-growth.

13. The medical device of claim 11, further comprising tines at the outer periphery of the tubular portion, the tines being oriented and configured to lodge the tubular portion against the tissue of the heart.

14. The medical device of claim 11, wherein the tubular portion comprises an upper loop portion and a lower loop portion with intermediate extensions therebetween.

15. The medical device of claim 14, wherein the intermediate portion extends from the lower loop portion of the tubular portion.

16. The medical device of claim 11, wherein the intermediate portion comprises multiple intermediate portions extending between different portions of the lower portion of the tubular portion.

17. The medical device of claim 11, wherein the intermediate portion comprises a first set of multiple lines extending in a first direction from the lower portion of the tubular portion and a second set of multiple lines extending in a second direction from the lower portion of the tubular portion, the first direction being substantially transverse to the second direction.

18. The medical device of claim 11, wherein the tubular portion is configured to self expand and bias against the tissue of the heart adjacently above the valve.

19. A medical device configured to be delivered with a catheter in a heart to minimize prolapse of a valve, the medical device comprising:
an intermediate portion configured to be positioned above a free-edge of the valve;
an anchor portion extending from the intermediate portion and configured to lodge against heart tissue above the valve; and
a plurality of tabs laterally extending from the intermediate portion and configured to abut against leaflets of the valve and substantially limit upward movement of the leaflets of the valve.

20. The medical device of claim 19, wherein the plurality of tabs extend downward and outward from opposite sides of the intermediate portion.

21. A method of reducing valve prolapse, the method comprising:
disposing a frame within the heart adjacent a valve; and
limiting movement of at least one leaflet of the valve with a portion of the frame.
